(19) United States
(12) Patent Application Publication
(10) Pub. No.: US 2024/0390334 A1
(43) Pub. Date: Nov. 28, 2024

(54) COMPOSITIONS AND METHODS FOR MITIGATING ALCOHOL LIVER DISEASE

(71) Applicants: Research Foundation of the City University of New York, NEW YORK, NY (US); Cornell University, lthaca, NY (US)

(72) Inventors: STEVEN TRASINO, New York, NY (US); LORRAINE GUDAS, New York, NY (US); XIAO-HAN TANG, NEW YORK, NY (US); MARTA MELIS, NEW YORK, NY (US)

(73) Assignees: Research Foundation of the City University of New York, NEW YORK, NY (US); Cornell University, Ithaca, NY (US)

(21) Appl. No.: 18/690,917

(22) PCT Filed: Sep. 14, 2022
(86) PCT No.: PCT/US22/76444

§ 371 Date: Mar. 11, 2024

Related U.S. Application Data
(60) Provisional application No. 63/244,289, filed on Sep. 15, 2021.

(51) Int. Cl. A61K 31/426; A61K 31/192; A61P 1/16

(52) U.S. Cl. CPC A61K 31/426; A61K 31/192; A61P 1/16

(57) ABSTRACT

Specifically, the invention relates to compositions and methods for treating liver ailments, such as compositions and methods for attenuating or ameliorating the development of alcoholic liver disease (ALD), hepatic steatosis, liver oxidative stress and/or hepatic lipogenesis associated with alcohol consumption in a subject by administering a retinoic acid receptor-beta (RARβ) agonist.

### GOVERNMENT FUNDING

This invention was made with government support under Grant Number 5SC2GM127206 awarded by the National Institute of General Medical Sciences and Grant Number R21AA027637 awarded by the National Institutes of Health. The government has certain rights in the invention.

### FIELD OF THE INVENTION

The invention relates to compositions and methods for treating liver ailments. Specifically, the invention relates to attenuating or ameliorating the development of liver ailments associated with alcohol consumption in a subject by administering a retinoic acid receptor-beta (RARβ) agonist.

### BACKGROUND OF THE INVENTION

Fatty liver disease occurs, in the form of alcoholic liver disease (ALD) induced by excessive alcohol consumption, or in the form of nonalcoholic fatty liver disease (NAFLD), primarily caused by obesity. Both ALD and NAFLD demonstrate a range of pathologic states, including steatosis, steatohepatitis, liver cirrhosis, and potentially development of hepatocellular carcinoma (HCC).

Because both the underlying causes and the therapeutic approaches are generally different, it is important to distinguish between alcoholic liver disease (ALD) and non-alcoholic liver disease (NAFLD). Although there are no FDA-approved treatments for ALD or NAFLD, the first-line therapeutic intervention for ALD is abstinence from alcohol and the administration of steroids. In contrast, the treatment for NAFLD is weight loss, exercise, and pharmacological correction of insulin resistance and/or reduction in tissue lipid levels (dyslipidemia).

Clinically, alcoholic liver disease (ALD) includes a threshold with respect to the duration of use and daily intake of alcohol.

Daily intake of alcohol for 10-12 years with doses in excess of 40-80 g/day for males and of 20-40 g/day for females is generally required to cause ALD, or daily drinking of 3-6 cans (12 oz each) of beer/day for males or 1.5-3 cans of beer/day for females for 10 years or longer will often result in ALD.

The risk for HCC increases fivefold with daily alcohol consumption of 80 g. In the presence of Hepatitis C infection, the HCC risk increases 20-fold; and a combination of both risk factors (alcohol consumption and Hepatitis C infection) increases the risk for HCC more than 100-fold.

Nonalcoholic fatty liver disease (NAFLD) develops in the absence of excessive alcohol consumption. Obesity is the most important risk factor for the development of this disorder, but NAFLD can also develop in patients with normal weight.

Vitamin A (all trans-retinol) and its derivatives, including retinal, retinyl-esters (RE), and all-trans retinoic acid (RA), are collectively known as retinoids. Primarily acting through its biologically active metabolite, RA, and cognate RA receptors (RAR α, β and γ), retinoids regulate the expression of thousands of genes critical for cellular differentiation, reproduction, immune function, and energy metabolism in cells and tissues, including the liver. The actions of retinoids, such as the potent, biologically active endogenous metabolite of vitamin A, all-trans retinoic acid (RA), are primarily mediated by binding to ligand-activated transcription factors, the retinoic acid receptors (RARs) α, β, and γ. When RARs bind the pan-agonist RA, they heterodimerize with retinoid X receptors (RXRs) α, β, and γ. Agonists that bind to different RAR receptors can be expected to have different effects because different RARs are known to serve different biological functions.

For more than three decades, numerous lines of evidence have shown that chronic alcohol intake promotes hepatic retinoid reduction and marked perturbations to whole-body retinoid status. If left unchecked, retinoid deficiency can contribute to the progression of liver damage and of ALD. For individuals who struggle with alcohol abuse, dietary vitamin A supplementation to correct this retinoid deficiency is not that effective because of the deleterious effects of EtOH on retinoid metabolism and other factors that contribute to retinoid and nutritional deficiencies in ALD. Moreover, when concomitantly administered with EtOH, dietary retinyl-palmitate or 3-carotene may be hepatoxic.

Accordingly, there remains an unmet need for therapeutic compositions and treatment methods capable of treating, preventing, or ameliorating alcoholic liver disease. There also remains an unmet need for therapeutic compositions and treatment methods that inhibits liver steatosis (fat accumulation), liver oxidative stress and/or hepatic lipogenesis associated with alcohol consumption.

### SUMMARY OF THE INVENTION

In one aspect, the invention provides methods of treating, preventing, or ameliorating alcoholic liver disease (ALD) in a subject, comprising administering to the subject a therapeutically effective amount of a selective retinoic acid receptor-β₂ (RARβ₂) agonist or a pharmaceutically acceptable salt, ester, amide, prodrug thereof, or a combination thereof. In an exemplary embodiment, said RARβ₂ agonist is AC261066.

In another aspect, the invention provides methods of inhibiting liver steatosis (fat accumulation) associated with alcohol consumption in a subject, comprising administering to the subject a therapeutically effective amount of a selective retinoic acid receptor-β2 (RARβ₂) agonist or a pharmaceutically acceptable salt, ester, amide, prodrug thereof, or a combination thereof. In an exemplary embodiment, said RARβ₂ agonist is AC261066.

In an additional aspect, the invention provides methods of inhibiting liver oxidative stress and/or hepatic lipogenesis associated with alcohol consumption in a subject, comprising administering to the subject a therapeutically effective amount of a selective retinoic acid receptor-β₂ (RARβ₂) agonist or a pharmaceutically acceptable salt, ester, amide, prodrug thereof, or a combination thereof. In an exemplary embodiment, said RARβ₂ agonist is AC261066.

In a further aspect, the invention provides a pharmaceutical formulation comprising a therapeutically effective amount of a selective retinoic acid receptor-β2 (RARβ₂) agonist and a pharmaceutically acceptable excipient. In an exemplary embodiment, said RARβ₂ agonist is AC261066.

Other features and advantages of this invention will become apparent from the following detailed description examples and figures. It should be understood, however, that the detailed description and the specific examples, while indicating various embodiments of the invention, are given by way of illustration only, since various changes and modifications within the spirit and scope of the invention will become apparent to those skilled in the art from the detailed description.

### BRIEF DESCRIPTION OF THE DRAWINGS

FIGS. 1A-1H: Treatment with AC261066 (AC261) mitigates hepatic steatosis in ethanol-fed mice. Representative images of hematoxylin and eosin livers; (FIGS. 1A-ID) (40×; inset a)-d) (200×) and (FIGS. 1E-1H) Oil-Red O stained liver sections; Scale Bar=100 μm.

FIGS. 2A-2C: Treatment with AC261066 (AC261) mitigates hepatic steatosis in ethanol-fed mice. (FIG. 2A) Quantification of liver histopathology micro- and macrovesicular steatosis, (FIG. 2B) Quantification of Oil Red O-stained liver sections, (FIG. 2C) Hepatic Triglycerides (mg/gram tissue). All data error bars represent±SD, with *p<0.05, **p<0.01. ***p<0.001, ns=not significant. ###p<0.001 and ####<0.0001 compared to pair-fed and +++p<0.001 and ++++<0.0001 compared to 5% EtOH-treated.

FIGS. 3A-3B: Treatment with AC261066 (AC261) preserves liver function in ethanol-fed mice. FIGS. 3A-3B show plasma activity of (U/L) of (FIG. 3A) aspartate aminotransferase (AST), and (FIG. 3B) alanine aminotransferase (ALT).

FIGS. 4A-4H AC261066 modulates centrilobular ALDH1A1, CYP2E1 and oxidative stress that is induced by ethanol. FIG. 4A and FIG. 4C are western blot gels, and FIG. 4B and FIG. 4D are western blot semi-quantitative histograms of hepatic expression of hepatic ALDH1A1 and CYP2E1 protein levels in pair-fed, in pair-fed+AC261, 5% EtOH-treated, and 5% EtOH+AC261. Histogram data points represent individual animal values of gel band densitometry expressed as the ratio of ALDH1A1/GAPDH and CYP2E1/β-actin. FIG. 4E: Representative immunohistochemistry images of hepatic ALDH1A1, CYP2E1, and 4-HNE. Magnification 100×, scale bars=100 m. FIGS. 4F-4H: Quantitation of ALDH1A1, CYP2E1 and 4-HNE IHC.

FIG. 5: Retinoic Acid Receptor β2 (RARβ₂) Agonist AC261066 mitigates ETOH induced changes in the mRNA levels of genes involved in lipogenesis (top panel) or catabolism of lipids (bottom panel). RNA-seq measurements of relative hepatic transcript levels of genes involved in lipid metabolism from Pair-fed mice and 5% ETOH-fed mice, respectively, with and without the RARβ₂ agonist AC261066. Errors bars represent±SD of (Pair-fed: n=6; ETOH-fed, n=10; ETOH-fed+AC261066, n=10; Pair-fed+AC261066, n=4) mouse groups. *p<0.05, **p<0.01, ***p<0.001, ****p<0.0001. FPKM, Fragments Per Kilobase of transcript per Million mapped reads.

### DETAILED DESCRIPTION OF THE INVENTION

The present subject matter may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

In the context of the present disclosure, by “about” a certain amount it is meant that the amount is within ±20% of the stated amount, or preferably within ±10% of the stated amount, or more preferably within ±5% of the stated amount.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

The present invention relates to treating liver ailments associated with alcohol consumption in a subject in need thereof, by administering to the subject a therapeutically effective amount of a retinoic acid receptor-beta (RARβ) agonist, in particular an RARβ₂ agonist. The present invention also relates to treating, preventing, or ameliorating alcoholic liver disease (ALD). The present invention also relates to methods of inhibiting liver steatosis (fat accumulation), liver oxidative stress and/or hepatic lipogenesis associated with alcohol consumption.

Various embodiments of the invention provide methods for treating, preventing, and ameliorating liver ailments associated with alcohol consumption and pharmaceutical formulations that comprise a retinoic acid receptor-beta (RARβ) agonist, more specifically, a selective RARβ₂ agonist. Such pharmaceutical formulations can be configured in various ways and in a variety of dosage forms, such as formulations for oral and peritoneal administration.

As used herein, the terms “decrease”, “reduce” and “diminish” are used interchangeably to refer to a negative change in the level, activity or function of a molecule, cell or organ. It is meant that the particular level, activity or function is lower by about 10%, about 25%, about 50%, about 75%, about 90%, about 1-fold, about 2-fold, about 5-fold, about 10-fold, about 25-fold, about 50-fold, or about 100-fold, or lower, when compared to a control.

As used herein, the terms “elevate”, “increase”, “improve” and “enhance” are used interchangeably to refer to a positive change in the level, activity or function of a molecule, cell or organ. It is meant that the particular level, activity or function is higher by about 10%, about 25%, about 50%, about 75%, about 90%, about 1-fold, about 2-fold, about 5-fold, about 10-fold, about 25-fold, about 50-fold, or about 100-fold, or higher, when compared to a control.

The retinoic acid receptor (RAR) is a type of nuclear receptor that is activated by all-trans retinoic acid (RA). There are three retinoic acid receptors (RAR), RARα, RARβ, and RARγ, encoded by the RARα, RARβ, RARγ genes, respectively. Each receptor isoform has several splice variants: four- for α, four- for β, and two- for γ.

RAR heterodimerizes with RXR and in the absence of ligand, the RAR/RXR dimer binds to hormone response elements known as retinoic acid response elements (RAREs) complexed with corepressor protein. Binding of agonist ligands to RAR results in dissociation of corepressor and recruitment of coactivator protein that, in turn, promotes transcription of the downstream target gene into mRNA and eventually protein.

The RARβ subtype consists of four known isoforms RARβ₁, RARβ₂, RARβ₃ and RARβ₄.

The ligand binding domains of the four isoforms are similar, while the variation between the isoforms is located within the proximal N-terminus, which encompasses the ligand-independent activation domain (AF-1).

It has been reported that the ligand binding domain, i.e., AF-2, of a given RAR isotype cooperates with the AF-1 domain in a promoter context manner. The AF-2 domains are conserved between the isoforms, the AF-1 domains are not. Using an RARβ (e.g., RARβ2) receptor-ligand crystal structure, various RARβ agonists have been designed and identified.

RARβ agonists include, but are not limited to, AC261066, AC55649, Tazarotene, Adapalene, 9-cis-retinoic acid and TTNPB. AC261066 and AC55649 are highly selective RARβ agonists. “Highly selective RARβ agonists” also include other agonists with a similar binding affinity to AC261066 or AC55649, e.g., at least 50% or greater, preferably 75% or greater, more preferably 90% or greater of the RARβ binding affinity of AC261066 or AC55649. “Highly selective RARβ₂ agonists” include agonists with a similar binding affinity as AC261066 or AC55649, e.g., at least 50% or greater, preferably 75% or greater, more preferably 90% or greater of the RARβ₂ binding affinity of AC261066 or AC55649. A highly selective RARβ (e.g., RARβ₂) agonist preferably has an affinity for RARβ (e.g., RARβ₂) greater than 6.00 pEC₅₀, more preferably greater than 6.50 pEC₅₀, more preferably greater than 7.00 pEC₅₀, more preferably greater than 7.50 pEC₅₀, more preferably greater than 7.75 pEC₅₀, and even more preferably greater than 8.00 pEC₅₀.

RARβ agonists include the fluorinated alkoxythiazoles previously described, such as:

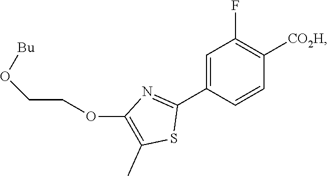

4′-Octyl-[1,1′-biphenyl]-4-carboxylic acid (65), Adapalene (67), BMS-231973, BMS-228987, BMS-276393, BMS-209641 (66), BMS-189453{4-[(1E)-2-(5,6-Dihydro-5,5-dimethyl-8-phenyl-2-naphthalenyl)ethenyl]-benzoic acid} (68), CD2019 (6-[4-methoxy-3-(1-methylcyclohexyl)phenyl]naphthalene-2-carboxylic acid), compounds described in WO2008/064136 and WO2007009083, each of which is incorporated herein by reference in its entirety, and tazarotene (ethyl 6-[2-(4,4-dimethyl-3,4-dihydro-2H-1-benzothiopyran-6-yl)ethynyl]pyridine-3-carboxylate). Structures of some RARβ agonists are provided below:

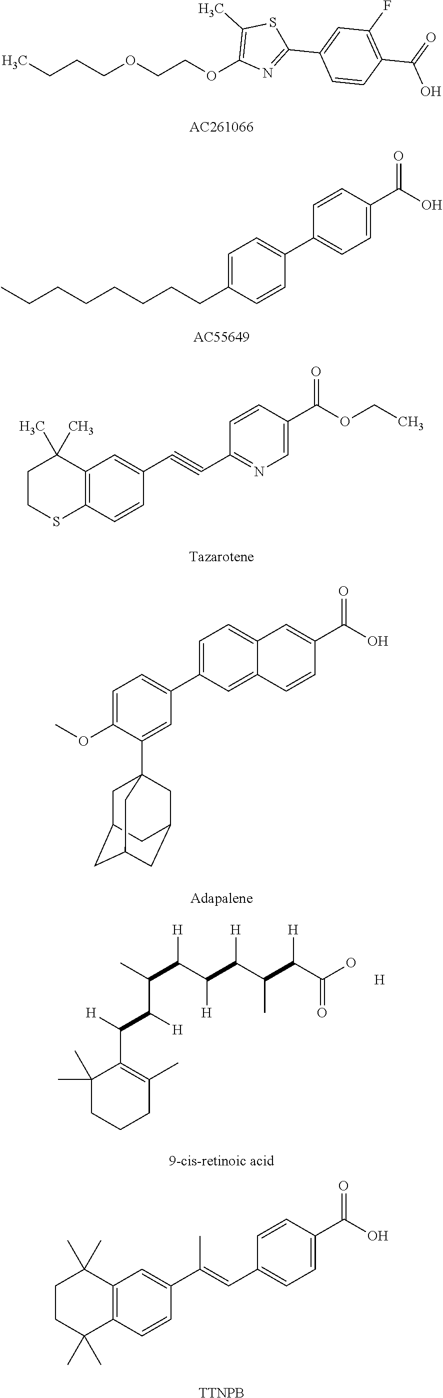

RARβ agonists also include those disclosed in published PCT patent application WO2008/064136, WO2007/009083 and published U.S. patent application US2009/0176837, each of which is incorporated herein by reference in its entirety. The highly selective RARβ agonists AC261066 and AC55649, are highly isoform-selective agonists for the human RARβ₂ receptors as described in Lund et al. (2005, J. Med. Chem., 48, 7517-7519), which is incorporated herein by reference in its entirety. In some embodiments, the RARβ₂ agonist is AC261066.

RARβ₂ receptor agonists may also be selected from the following compounds or an ester thereof:

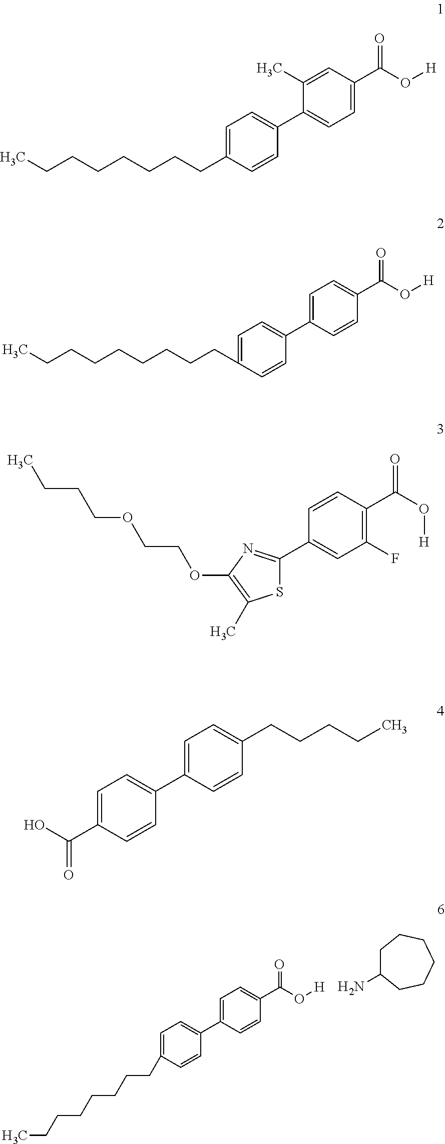

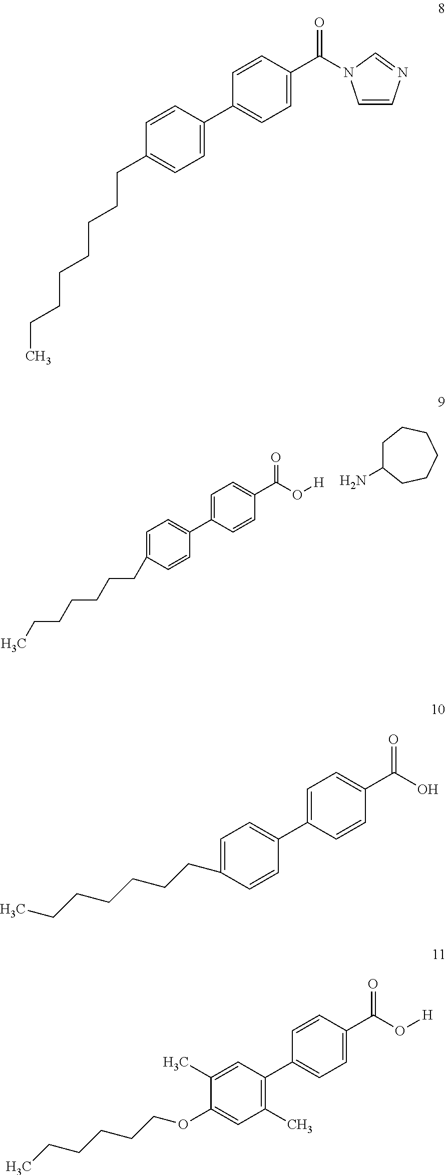

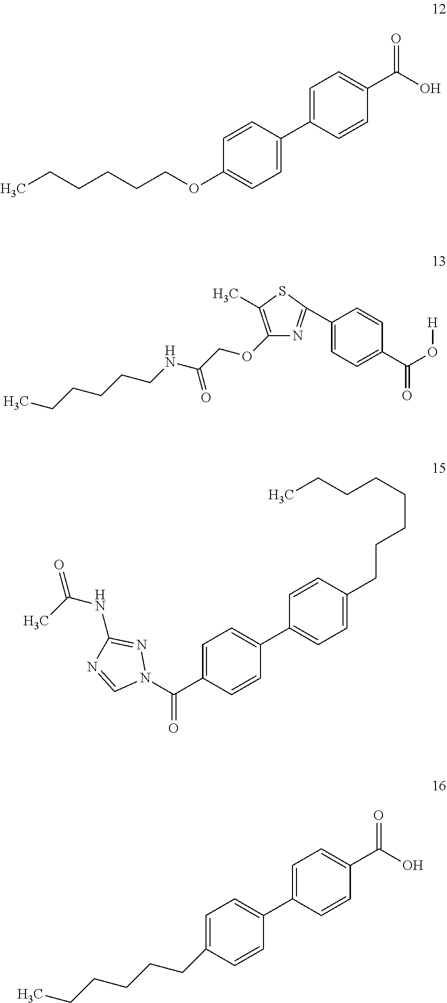 [SMILES: CCCCCCOc1ccc(-c2ccc(C(=O)O)cc2)cc1 ; CCCCCCNC(=O)COc1nc(-c2ccc(C(=O)O)cc2)sc1C ; CCCCCCCCc1ccc(-c2ccc(C(=O)n3cnc(NC(C)=O)n3)cc2)cc1 ; CCCCCCc1ccc(-c2ccc(C(=O)O)cc2)cc1]

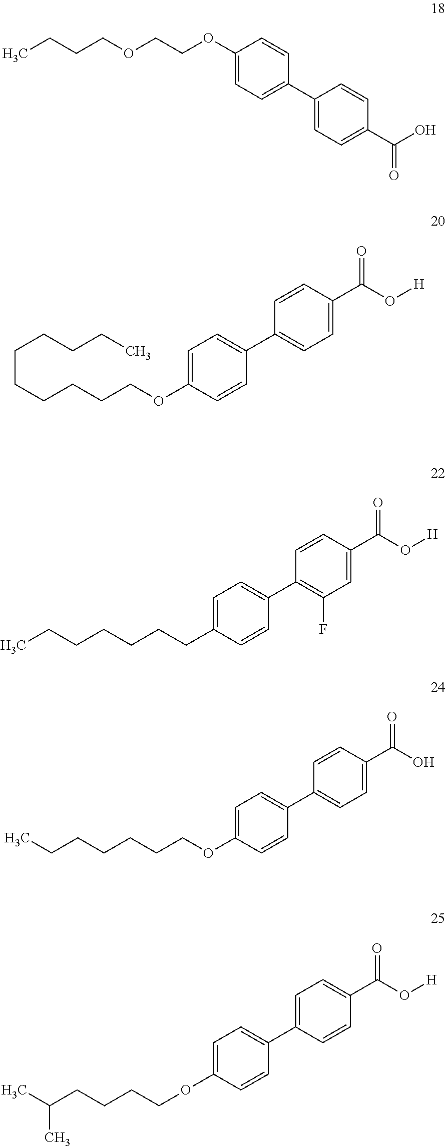 [SMILES: CCCCOCCOc1ccc(-c2ccc(C(=O)O)cc2)cc1 ; CCCCCCCCCCOc1ccc(-c2ccc(C(=O)O)cc2)cc1 ; CCCCCCCc1ccc(-c2ccc(C(=O)O)cc2F)cc1 ; CCCCCCCOc1ccc(-c2ccc(C(=O)O)cc2)cc1 ; CC(C)CCCCOc1ccc(-c2ccc(C(=O)O)cc2)cc1]

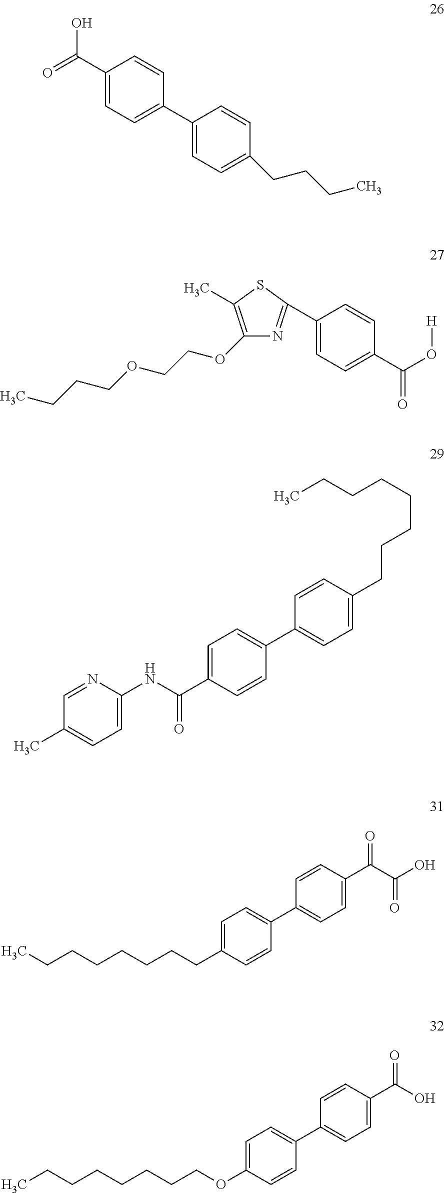

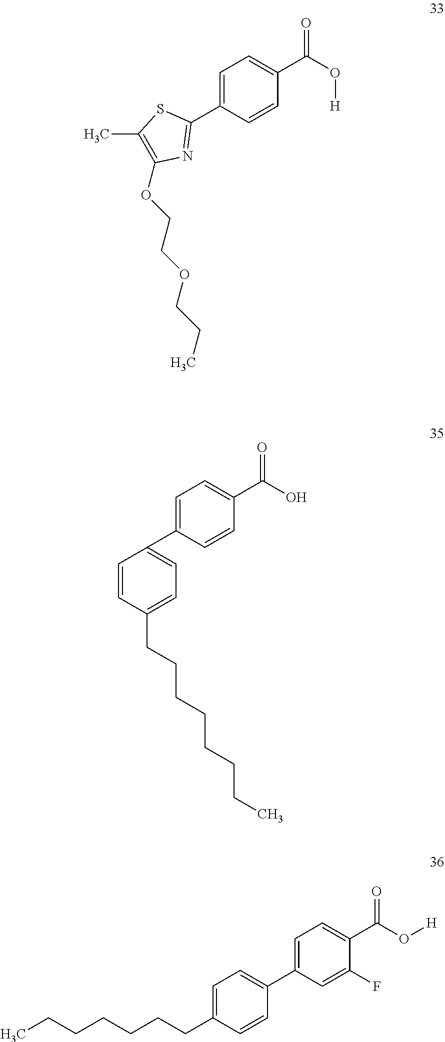 [SMILES: CCCOCCOc1nc(-c2ccc(C(=O)O)cc2)sc1C ; CCCCCCCCc1ccc(-c2ccc(C(=O)O)cc2)cc1 ; CCCCCCCc1ccc(-c2ccc(C(=O)O)c(F)c2)cc1]

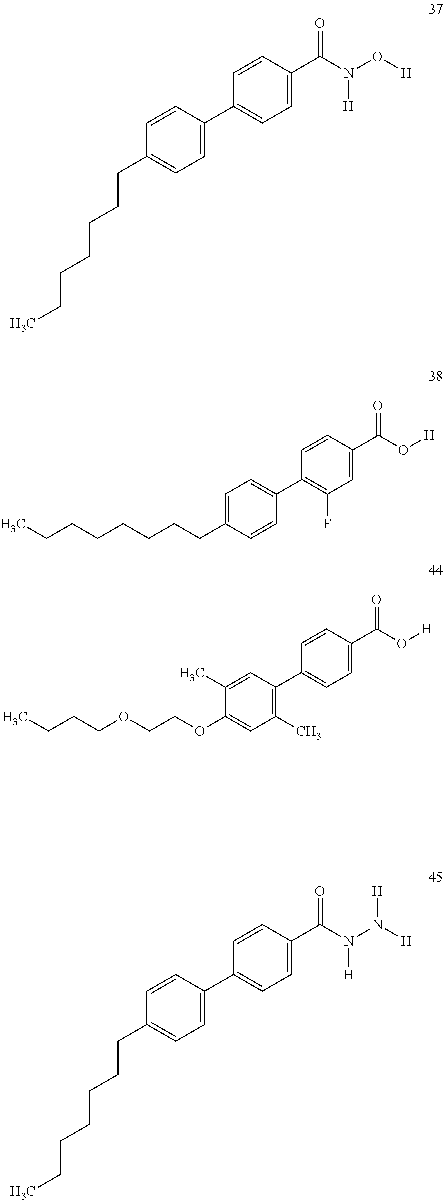 [SMILES: CCCCCCCc1ccc(-c2ccc(C(=O)NO)cc2)cc1 ; CCCCCCCCc1ccc(-c2ccc(C(=O)O)cc2F)cc1 ; CCCCOCCOc1cc(C)c(-c2ccc(C(=O)O)cc2)cc1C ; CCCCCCCc1ccc(-c2ccc(C(=O)NN)cc2)cc1]

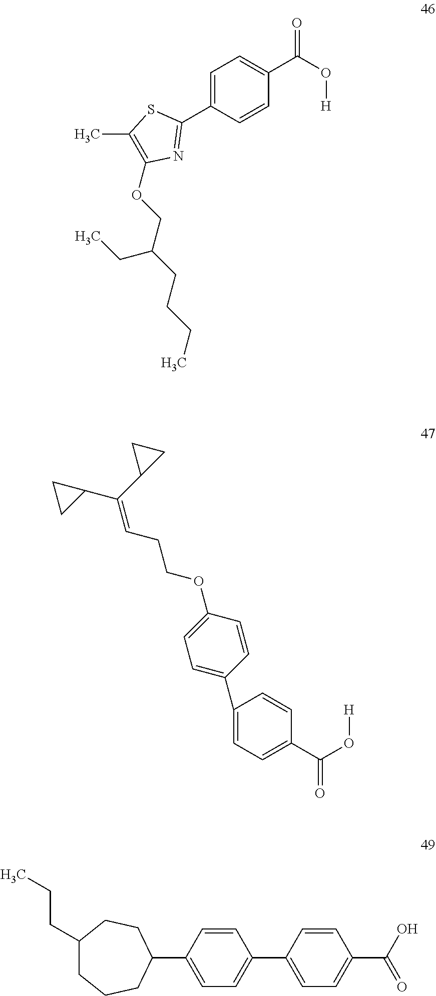 [SMILES: CCCCC(CC)COc1nc(-c2ccc(C(=O)O)cc2)sc1C ; O=C(O)c1ccc(-c2ccc(OCCC=C(C3CC3)C3CC3)cc2)cc1 ; CCCC1CCCC(c2ccc(-c3ccc(C(=O)O)cc3)cc2)CC1]

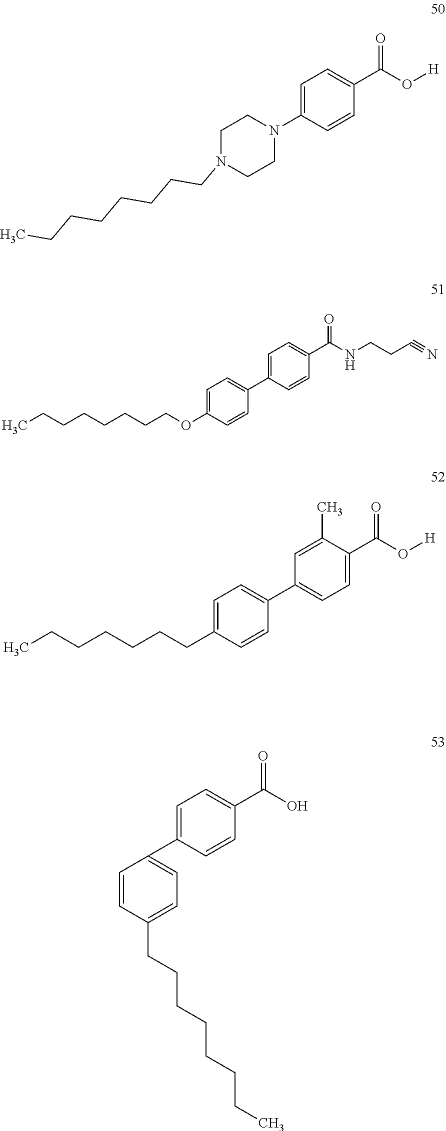 [SMILES: CCCCCCCCN1CCN(c2ccc(C(=O)O)cc2)CC1 ; CCCCCCCCOc1ccc(-c2ccc(C(=O)NCCC#N)cc2)cc1 ; CCCCCCCc1ccc(-c2ccc(C(=O)O)c(C)c2)cc1 ; CCCCCCCCc1ccc(-c2ccc(C(=O)O)cc2)cc1]

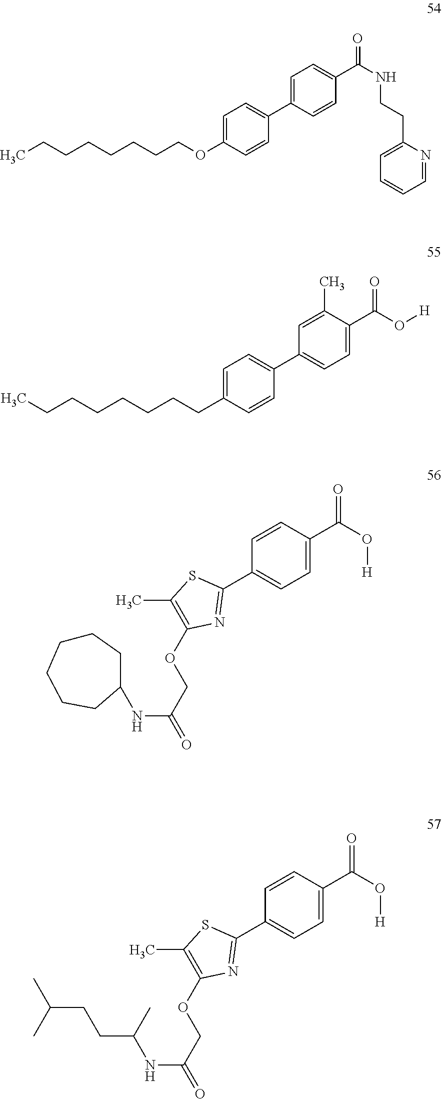 [SMILES: CCCCCCCCOc1ccc(-c2ccc(C(=O)NCCc3ccccn3)cc2)cc1 ; CCCCCCCCc1ccc(-c2ccc(C(=O)O)c(C)c2)cc1 ; Cc1sc(-c2ccc(C(=O)O)cc2)nc1OCC(=O)NC1CCCCCC1 ; Cc1sc(-c2ccc(C(=O)O)cc2)nc1OCC(=O)NC(C)CCC(C)C]

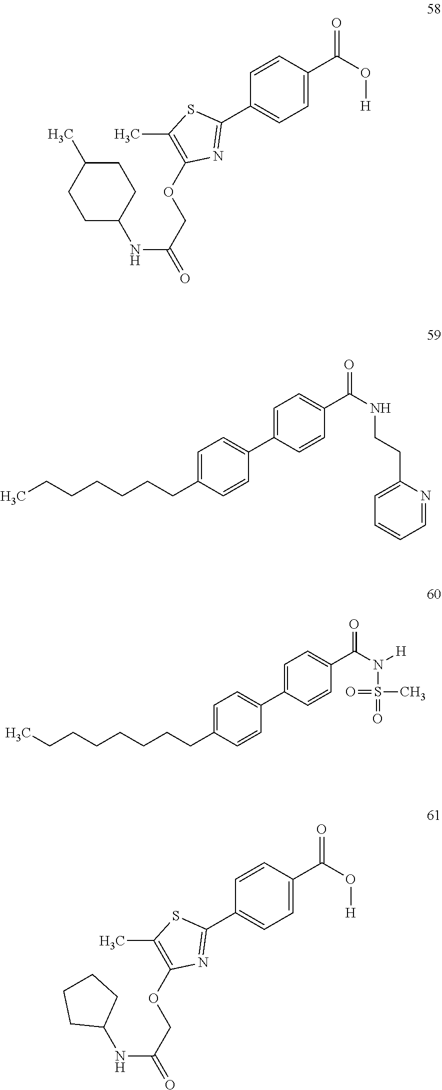 [SMILES: Cc1sc(-c2ccc(C(=O)O)cc2)nc1OCC(=O)NC1CCC(C)CC1 ; CCCCCCCc1ccc(-c2ccc(C(=O)NCCc3ccccn3)cc2)cc1 ; CCCCCCCCc1ccc(-c2ccc(C(=O)NS(C)(=O)=O)cc2)cc1 ; Cc1sc(-c2ccc(C(=O)O)cc2)nc1OCC(=O)NC1CCCC1]

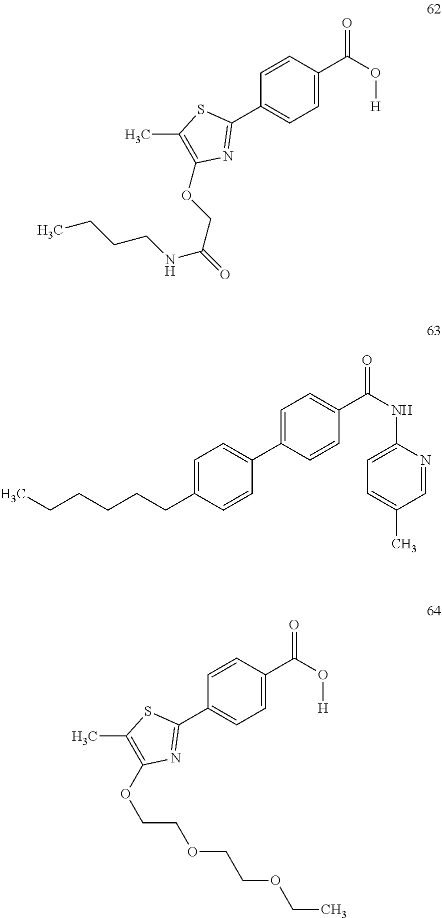 [SMILES: CCCCNC(=O)COc1nc(-c2ccc(C(=O)O)cc2)sc1C ; CCCCCCc1ccc(-c2ccc(C(=O)Nc3ccc(C)cn3)cc2)cc1 ; CCOCCOCCOc1nc(-c2ccc(C(=O)O)cc2)sc1C]

The functional receptor assay, receptor selection and amplification may be performed as described in WO2007/009083, which is incorporated herein by reference in its entirety. For example, Technology (R-SAT) may be used to investigate the pharmacological properties of known and novel RARβ agonists useful for the present invention. R-SAT is disclosed, for example, in U.S. Pat. Nos. 5,707,798, 5,912,132, and 5,955,281, Piu et al., 2006. Beta Arrestin 2 modulates the activity of Nuclear Receptor RARβ₂ through activation of ERK2 kinase, Oncogen, 25(2):218-29 and Burstein et al., 2006, Integrative Functional Assays, Chemical Genomics and High Throughput Screening: Harnessing signal transduction pathways to a common HTS readout, Curr Pharm Des, 12(14): 1717-29, all of which are hereby incorporated herein by reference in their entireties, including any drawings.

The relevant RARβ₂ receptor modulating activities of the above compounds are described in WO2007/009083, which is incorporated by reference herein in its entirety.

In one aspect, the invention provides method of treating, preventing, or ameliorating Alcoholic Liver Disease (ALD) in a subject, comprising administering to the subject a therapeutically effective amount of a selective retinoic acid receptor-β2 (RARβ₂) agonist, e.g., AC261066 or AC55649, or a pharmaceutically acceptable salt, ester, amide, prodrug thereof, or a combination thereof. In an exemplary embodiment, said RARβ agonist is AC261066.

In another aspect, the invention provides a method of inhibiting liver steatosis associated with alcohol consumption in a subject, comprising administering to the subject a therapeutically effective amount of a selective retinoic acid receptor-β2 (RARβ₂) agonist, e.g., AC261066 or AC55649, or a pharmaceutically acceptable salt, ester, amide, prodrug thereof, or a combination thereof.

In an additional aspect, the invention provides a method of inhibiting liver oxidative stress and/or hepatic lipogenesis associated with alcohol consumption in a subject, comprising administering to the subject a therapeutically effective amount of a selective retinoic acid receptor-β2 (RARβ₂) agonist, e.g., AC261066 or AC55649, or a pharmaceutically acceptable salt, ester, amide, prodrug thereof, or a combination thereof.

In some embodiments, the RARβ₂ agonist is administered chronically. In some embodiments, the RARβ₂ agonist is administered acutely. In some embodiments, the RARβ₂ agonist is administered after a diagnosis of ALD in the subject. In some embodiments, the RARβ₂ agonist is administered before a diagnosis of ALD in a subject at risk of developing ALD. In some embodiments, the RARβ₂ agonist is administered orally, intravenously, subcutaneously, sublingually, buccally, nasally, intraarterially, intracardially, transdermally, transmucosally, intramuscularly, intraperitoneally, topically, or a combination thereof. In some embodiments, the RARβ₂ agonist is administered orally. In some embodiments, the RARβ₂ agonist is administered parenterally. In some embodiments, the ALD is associated with reduced liver vitamin A levels.

In some aspects of the pharmaceutical formulations and methods provided herein, the RARβ agonist is a synthetic selective retinoic acid β₂-receptor (RARβ₂) agonist or a pharmaceutically acceptable salt, ester, amide, prodrug thereof, or a combination thereof.

In certain embodiments, the RARβ₂ agonist is AC261066 having a structure of Formula 1:

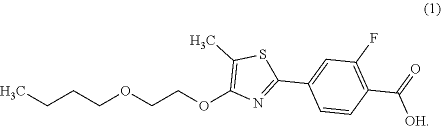 [SMILES: CCCCOCCOc1nc(-c2ccc(C(=O)O)c(F)c2)sc1C]

In other embodiments, the RARβ₂ agonist is AC55649 having a structure of Formula 2:

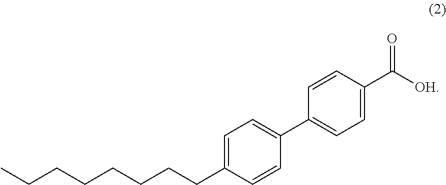 [SMILES: CCCCCCCCc1ccc(-c2ccc(C(=O)O)cc2)cc1]

In alternate embodiments, the RARβ agonist is tazarotene having a structure of Formula 3:

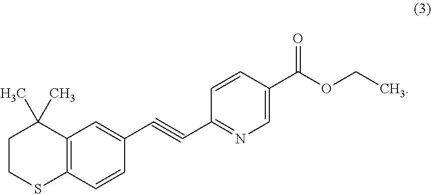 [SMILES: CCOC(=O)c1ccc(C#Cc2ccc3c(c2)C(C)(C)CCS3)nc1]

In another aspect, the RARβ agonist is adapalene having a structure of Formula 4:

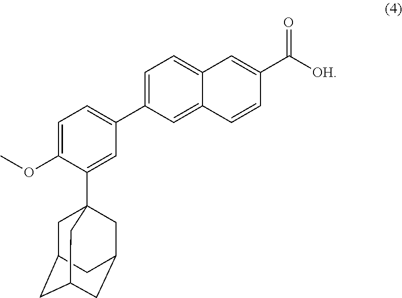 [SMILES: COc1ccc(-c2ccc3cc(C(=O)O)ccc3c2)cc1C12CC3CC(CC(C3)C1)C2]

In still another aspect, the RARβ agonist is 4-[(E)-2-(5,6,7,8-Tetrahydro-5,5,8,8-tetramethyl-2-naphthalenyl)-1-propenyl]benzoic acid (TTNPB) having a structure of Formula 5:

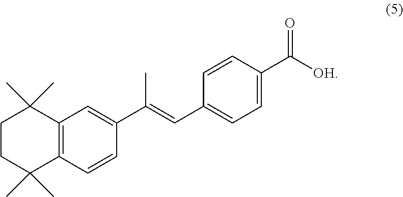 [SMILES: C/C(=C\c1ccc(C(=O)O)cc1)c1ccc2c(c1)C(C)(C)CCC2(C)C]

In certain embodiments, the RARβ2 agonist may be administered three times daily. In other embodiments, the RARβ₂ agonist may be administered in an amount from about 30 mg to about 200 mg per day.

In other aspects, the RARβ₂ agonist may be administered at a concentration of from about 0.1 mg to about 10 mg per 100 mL. The RARβ2 agonist also may be administered at a concentration from about 1 mg to about 3 mg per 100 mL.

In various embodiments, the RARβ₂ agonist may be administered orally. Alternatively, the RARβ agonist may be administered parenterally.

Parenteral administration includes intravenous, subcutaneous, sublingual, buccal, nasal, intraarterial, intracardiac, intraarticular, transdermal, transmucosal, intramuscular, intraperitoneal, ophthalmic and/or topical administration.

In other aspects, the presently provided methods further comprise administering to the subject a second therapeutic agent. The second therapeutic agent may be a second retinoic acid receptor-beta (RARβ) agonist. The second administered RARβ agonist may be an RARβ₂ agonist, for example, AC261066 having a structure of Formula 1:

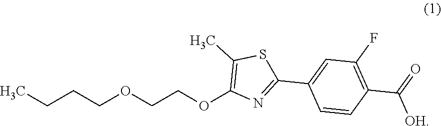 [SMILES: CCCCOCCOc1nc(-c2ccc(C(=O)O)c(F)c2)sc1C]

In an additional embodiment, the second administered RARβ agonist may be an RARβ₂ agonist, for example, AC55649 having a structure of Formula 2:

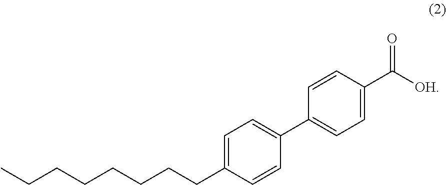 [SMILES: CCCCCCCCc1ccc(-c2ccc(C(=O)O)cc2)cc1]

In another aspect, the invention provides methods for inhibiting liver steatosis (fat accumulation), liver oxidative stress and/or hepatic lipogenesis associated with alcohol consumption in a subject in need thereof, comprising administering to the subject a therapeutically effective amount of a retinoic acid receptor-beta (RARβ) agonist. In certain embodiments, the RARβ agonist is a synthetic selective retinoic acid β₂-receptor (RARβ₂) agonist or a pharmaceutically acceptable salt, ester, amide, prodrug thereof, or a combination thereof. The RARβ₂ agonist may have a structure of Formula (1), as shown above. Alternatively, the RARβ₂ agonist may have a structure of Formula (2), as shown above. In additional embodiments, the RARβ agonist may have a structure of any one of Formula (3), Formula (4), or Formula (5), each of which is shown above.

In additional aspects of the methods for inhibiting liver steatosis (fat accumulation), liver oxidative stress and/or hepatic lipogenesis associated with alcohol consumption in a subject in need thereof, the method further comprises administering to the subject a second therapeutic agent. The second therapeutic agent may be a second retinoic acid receptor-beta (RARβ) agonist, and, in particular, the second retinoic acid receptor-beta (RARβ) agonist may be an RARβ2 agonist having a structure of Formula 1, as shown above, or an RARβ₂ agonist having a structure of Formula 2, as shown above.

The pharmaceutical formulation may comprise the RARβ₂ agonist in an amount of from about 30 mg to about 200 mg. In certain aspects, the pharmaceutical formulation may comprise the RARβ₂ agonist in an amount of from about 10 mg to about 100 mg. In various embodiments, the pharmaceutical formulation may comprise the RARβ₂ agonist an amount of from about 10 mg to about 100 mg. Alternatively, the pharmaceutical formulation may comprise the RARβ₂ agonist in an amount of from about 10 mg to about 67 mg. The pharmaceutical formulation may comprise the RARβ₂ agonist in a concentration of from about 0.1 mg to about 10 mg per 100 mL. In other aspects, the pharmaceutical formulation may comprise the RARβ₂ agonist in a concentration from about 1 mg to about 3 mg per 100 mL.

The pharmaceutical formulation comprising the RARβ agonist, in particular, an RARβ₂ agonist, may be formulated with a pharmaceutically acceptable excipient for oral administration. In an alternate aspect, the pharmaceutical formulation may be formulated with a pharmaceutically acceptable excipient for parenteral administration. In other aspects, the pharmaceutical formulation may be formulated with a pharmaceutically acceptable excipient for intravenous, subcutaneous, sublingual, buccal, nasal, intraarterial, intracardiac, intraarticular, transdermal, transmucosal, intramuscular, intraperitoneal, ophthalmic, or topical administration.

In a further aspect, the invention provides a pharmaceutical formulation comprising a therapeutically effective amount of a retinoic acid receptor-β2 (RARβ₂) agonist and a pharmaceutically acceptable excipient.

As used herein, the term “pharmaceutically acceptable excipient” refers to those excipients which are, within the scope of sound medical judgment, suitable for use in contact with the tissues of humans and lower animals without undue toxicity, irritation, allergic response, and the like, and are commensurate with a reasonable benefit/risk ratio.

The term “pharmaceutically acceptable excipient” includes any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000, incorporated herein by reference in its entirety, specifically incorporated by reference in its entirety are disclosures of various excipients and carriers used in formulating pharmaceutically acceptable compositions and known techniques for the preparation thereof.

The pharmaceutical compositions of the invention may be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions, or solutions. Formulations may optionally contain excipients, including but not limited to, solvents, diluents, and other liquid vehicles, dispersion or suspension aids, surface active agents, pH modifiers, isotonic agents, thickening or emulsifying agents, stabilizers and preservatives, solid binders, lubricants, and the like, as suited to the particular dosage form desired.

The RARβ agonist, and, in particular, a RARβ₂ agonist, may be administered by any suitable method known to one skilled in the art. For example, an RARβ agonist, and, in particular, a RARβ₂ agonist, may be administered orally or parenterally.

It is to be understood by one of skill in the art that the methods of treatment and/or prevention comprising administering a RARβ agonist provided herein for the treatment and/or prevention of one or more indications as described herein also include: the use of a RARβ agonist provided herein in the manufacture of a medicament for the treatment and/or prevention of one or more indications as described herein; and the use of a RARβ agonist provided herein for the treatment and/or prevention of one or more indications as described herein.

The term “parenteral” as used herein includes subcutaneous, intravenous, intramuscular, intra-articular, intra-synovial, intrasternal, intrathecal, intrahepatic, intralesional and intracranial injection or infusion techniques. The pharmaceutical compositions and formulations may be administered orally, intravenously, or subcutaneously. The formulations of the invention may be designed to be short-acting, fast-releasing, or long-acting. Still further, compounds may be administered locally, rather than by systemic means.

Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers. Besides inert diluents, the oral compositions can also include adjuvants such as wetting agents, emulsifying and suspending agents, sweetening, flavoring, and perfuming agents.

Injectable pharmaceutical formulations comprising an RARβ agonist, specifically an RARβ₂ agonist, for example, sterile injectable aqueous or oleaginous suspensions may be formulated according to the known art using suitable dispersing or wetting agents and suspending agents. The sterile injectable pharmaceutical formulation may also be a sterile injectable solution, suspension or emulsion in a nontoxic parenterally acceptable diluent or solvent, for example, as a solution in 1,3-butanediol. Among the pharmaceutically acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed, including but not limited to, synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

The RARβ agonist, in particular, an RARβ2 agonist, of the present invention may be used in combination with a second drug or therapeutic agent, wherein the second drug or second therapeutic agent is administered in a therapeutically effective amount to a subject in need thereof. The second therapeutic agent may be a second retinoic acid receptor-beta (RARβ) agonist.

All patents and literature references cited in the present specification are hereby incorporated by reference in their entirety.

### Examples

This Example addresses two challenges in the nutritional and clinical management of early stages of ALD—preserving hepatic retinoid status and preventing progression of liver damage. Evidence suggests that synthetic, micronutrient analogues could be an alternative to dietary approaches for resolving micronutrient deficiencies and pathology associated with ALD, because such analogues are not negatively impacted by EtOH status. Thus, it was sought to determine if a synthetic, orally available agonist for RARβ₂, AC261066 (AC261), could mitigate endogenous hepatic retinoid loss and protect against progression of ALD. Here the effects of EtOH and AC261 on retinoid status and RA-signaling was examined in a model of early ALD in which alcohol has deleterious effects on hepatic retinoid metabolism.

### Materials and Methods:

Liquid Ethanol Diet. Male C57BL/6J mice (8-9-weeks-old) (n=30) were purchased from Jackson Laboratory. Animals were housed in standard cages with 2-mice per cage and fed a standard vivarium chow (rodent diet #2014, Harlan-Teklad, Madison, WI). After acclimation to liquid feeding, mice were switched to the Lieber-DeCarli-liquid alcohol diet for 3 weeks using the following groups: 1) Control diet-fed mice (aka Pair-fed (PF), n=6), 2) Pair-fed mice+RARβ₂ specific agonist AC261066 (Tocris #4046), at 10 mg/kg/day [Pair-fed+AC261, (n=4)] (PF-AC), 3) Alcohol-treated (5% ethanol (EtOH) v/v plus vehicle [0.1% DMSO], n=10) (EtOH), and iv) 4) EtOH+AC261, [10 mg/kg/day], n=10)] (EtOH-AC).

### Results:

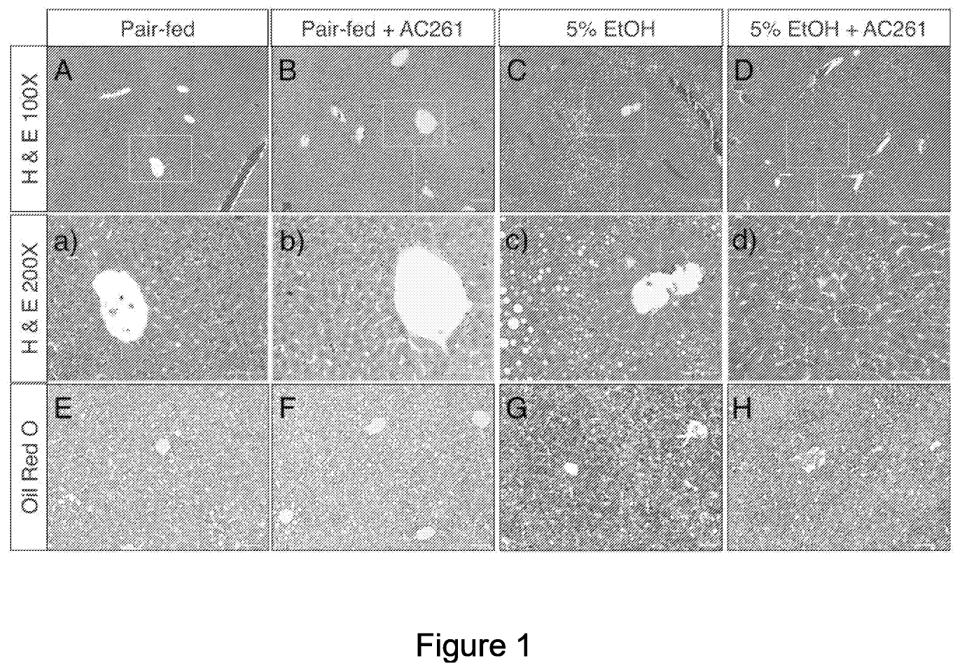

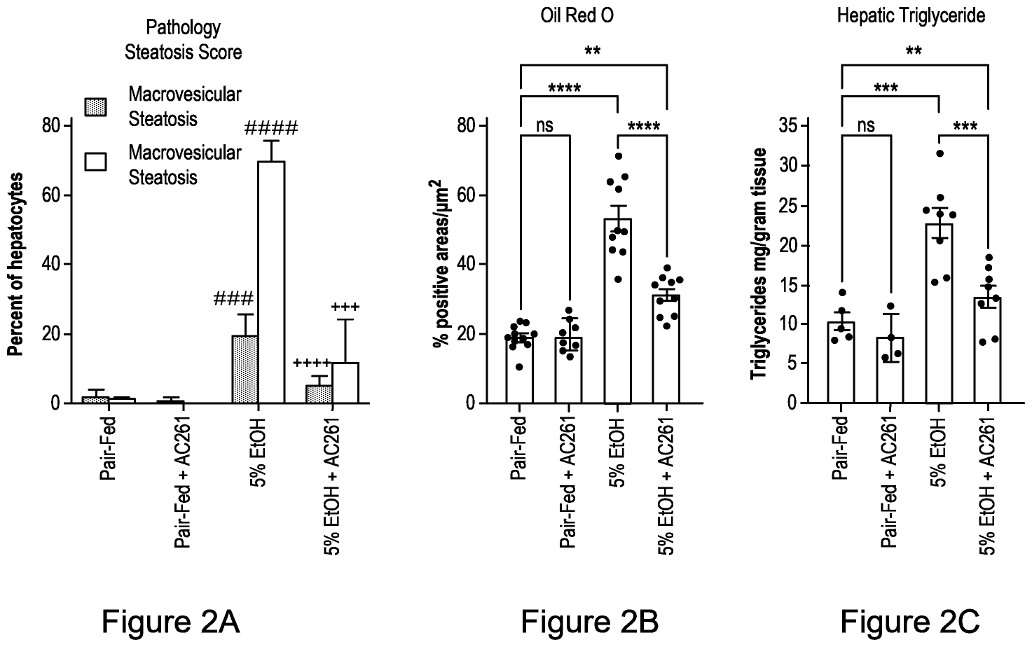

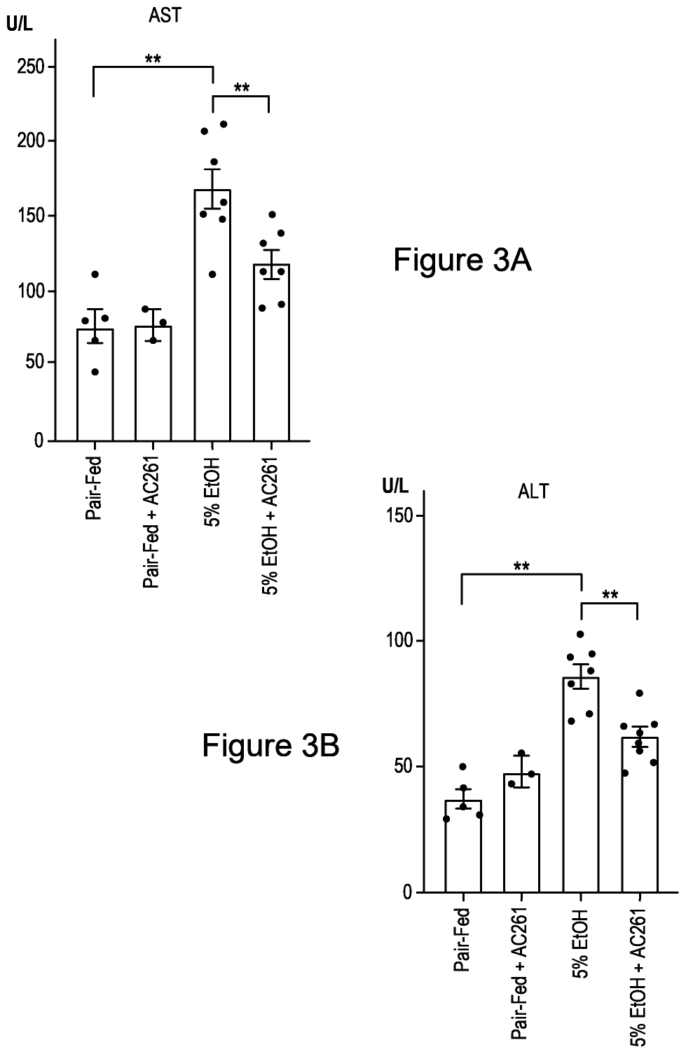

Treatment with AC261066 (AC261) mitigates hepatic steatosis and preserves liver function in ethanol-fed mice. Consistent with early stages of rodent and human ALD, histopathology evaluation showed that 3 weeks of chronic EtOH resulted in fatty liver in pan-lobular regions, but predominantly distributed in zones 2 and 3, which coincide with the mid-zonal and centrilobular regions of the liver, respectively (FIG. 1C vs A, arrows). Moreover, consistent with early-ALD, no evidence was found of hepatic fibrosis, as assessed by Picrosirius Red staining. Compared to pair-fed (PF), an almost 10-fold increase was detected in macrovesicular steatosis (FIG. 1 c) vs. a), red arrow, FIG. 2A), and a 70-fold increase in microvesicular steatosis (FIG. 1 c) vs. a) yellow arrow, FIG. 2A) in EtOH mice. Similarly, Oil red O lipid-staining (FIG. 1G vs. E; FIG. 2B), hepatic triglyceride levels (FIG. 2C). Plasma activity levels of aspartate aminotransferase (AST) and alanine aminotransferase (ALT), markers of liver injury (28), increased 2.1-fold, and 2.4-fold in EtOH compared to PF mice (FIGS. 3A-3B). In contrast, it was found that, compared to EtOH, EtOH-AC261 mice had 72% and 82% reductions in macrovesicular and microvesicular steatosis, respectively (FIG. 1D, d) vs. C, c), red and yellow arrows; FIG. 2A). Consistent with this, Oil red O lipid-staining, and hepatic triglycerides were reduced by 41% and 35% respectively, in EtOH-AC261 versus EtOH mice (FIG. 1H vs. G; FIG. 2B-2C). Almost 30% reductions in plasma activities of AST and ALT in EtOH-AC261 compared to EtOH mice was also detected (FIGS. 3A-3B). No evidence was found of changes to hepatic steatosis, triglyceride levels, fibrosis, or liver injury in PF+AC261 mice (FIG. 1; FIG. 2; FIG. 3). Additionally, through the entire length of the study, there were no group differences in daily food intake and body weights, and no differences in blood alcohol levels between EtOH and EtOH-AC261 mice. Together, these data show that 3 weeks of concomitant administration of AC261 with EtOH protected mice against hepatic steatosis and liver injury due to chronic EtOH intake.

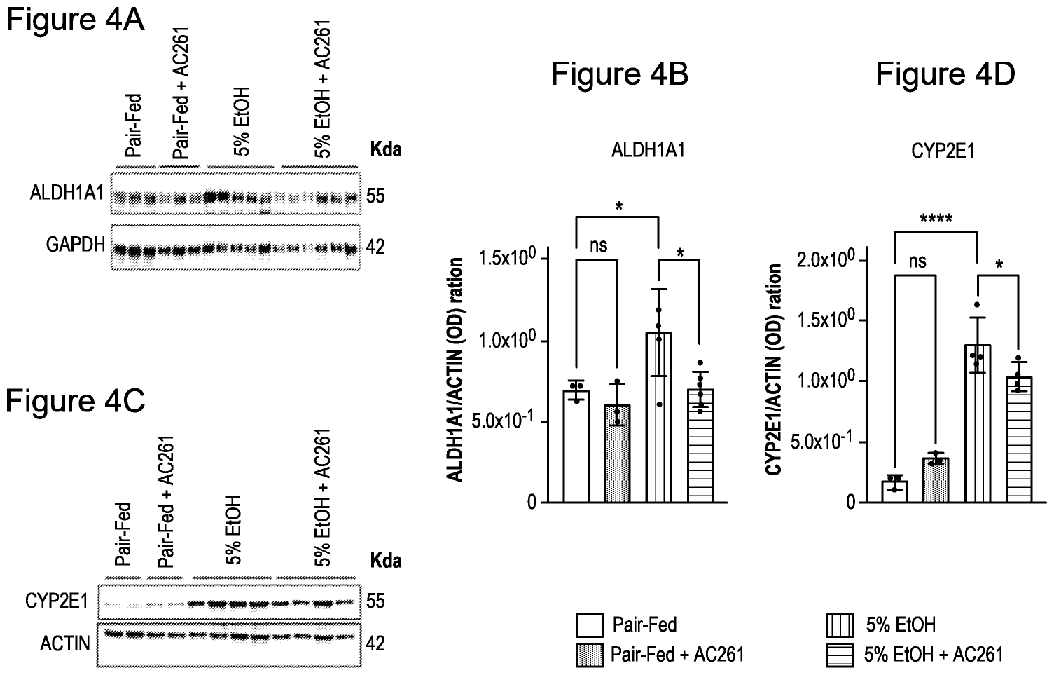

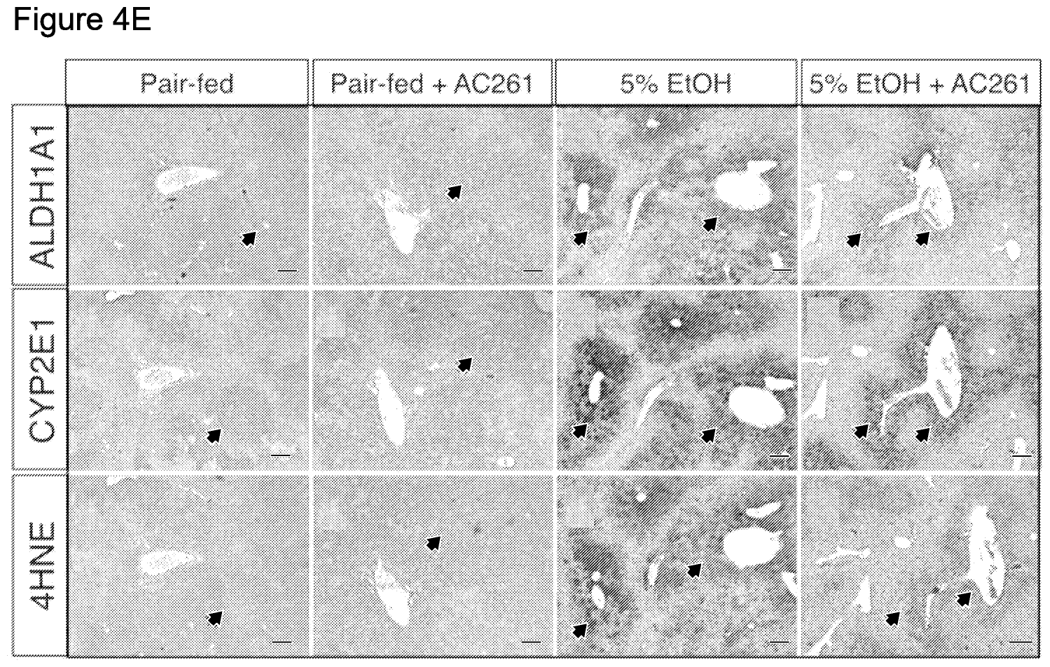

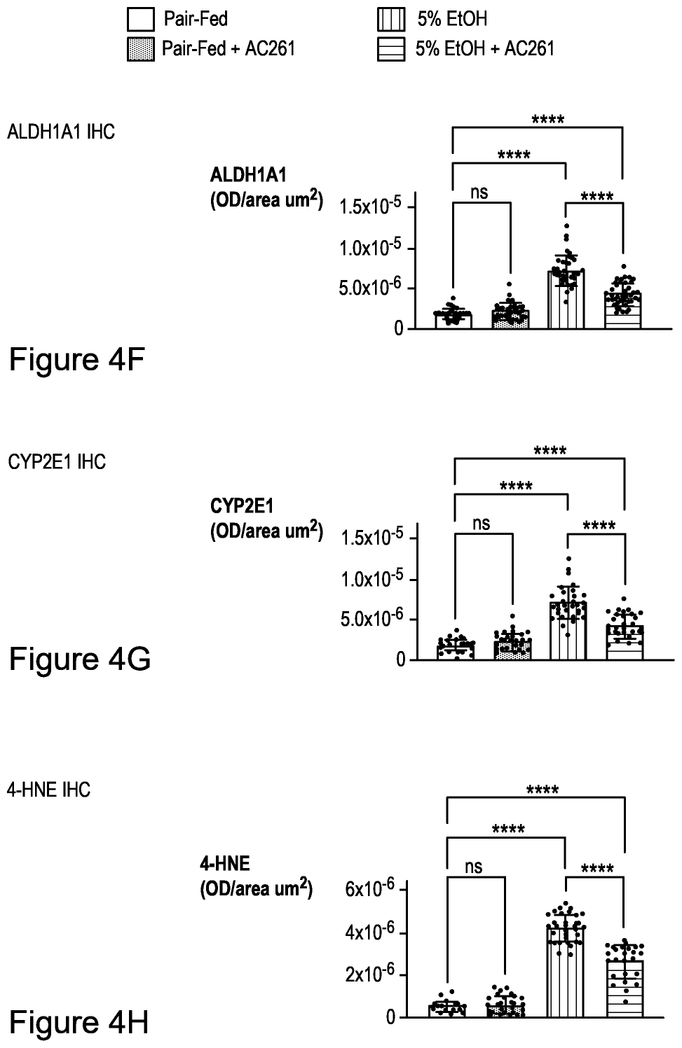

AC261066 and ethanol modulate centrilobular protein expression of ALDH1A1, CYP2E1, and Oxidative Stress. ALDH1A1 is one of the rate-limiting enzymes in the oxidation of retinaldehyde to RA and a key contributor to hepatic RA synthesis, as mice lacking ALDH1A1 show reduced hepatic levels of RA. However, ALDH1A1 also oxidizes acetaldehyde to acetate. Therefore, hepatic protein levels of ALDH1A1 were measured next, as it was the only ALDH1A isoform to increase in EtOH compared to PF mice. By western blotting and IHC, it was found that total hepatic ALDH1A1 protein levels increased by approximately 50%, and that, compared to PF mice, ALDH1A1 immunoreactivity was predominantly in zone 2-3, centrilobular regions in EtOH mice (FIG. 4E, arrows, F). In contrast, and consistent with reductions in mRNA levels, ALDH1A1 total protein, and zone 2-3, centrilobular ALDH1A1 protein immunoreactivity were reduced by 32% and 40% in EtOH-AC compared to EtOH mice (FIG. 4E, arrows, F). No changes were detected in hepatic ALDH1A1 protein in PF-AC compared to PF mice (FIG. 4A, B, E, F). Hepatic centrilobular regions are the first to develop liver damage in early stages of ALD, which is largely driven by CYP2E1-mediated EtOH metabolism and generation of reactive oxygen species (ROS). Therefore, given that high levels of ALDH1A1 were specifically detected only in zone 2-3, centrilobular regions, hepatic CYP2E1 protein levels were next measured by western blot and IHC, and by IHC, 4-HNE, as an indirect readout of oxidative stress. Total CYP2E1 protein levels were increased by almost 8-fold in EtOH compared to PF mice. However, by IHC it was also found that CYP2E1 and 4-HNE staining showed striking overlap with ALDH1A1 in zone 2-3, centrilobular regions in EtOH mice (FIG. 4E, arrows, F, G, H). In contrast, livers of EtOH-AC261066 mice showed approximately 31% and 39% reductions in hepatic CYP2E1 protein levels and zone 2-3 centrilobular CYP2E1 protein levels, respectively (FIG. 4E, arrows, G). Consistent with this, hepatic levels of 4-HNE (FIG. 4E, H), decreased by 33% and 36%, respectively, in EtOH-AC261 compared to EtOH mice. Together, this data suggests that in response to 3 weeks of chronic EtOH intake, hepatic centrilobular regions with high levels of oxidative stress from CYP2E1-EtOH metabolism may also have high levels of RA synthesis.

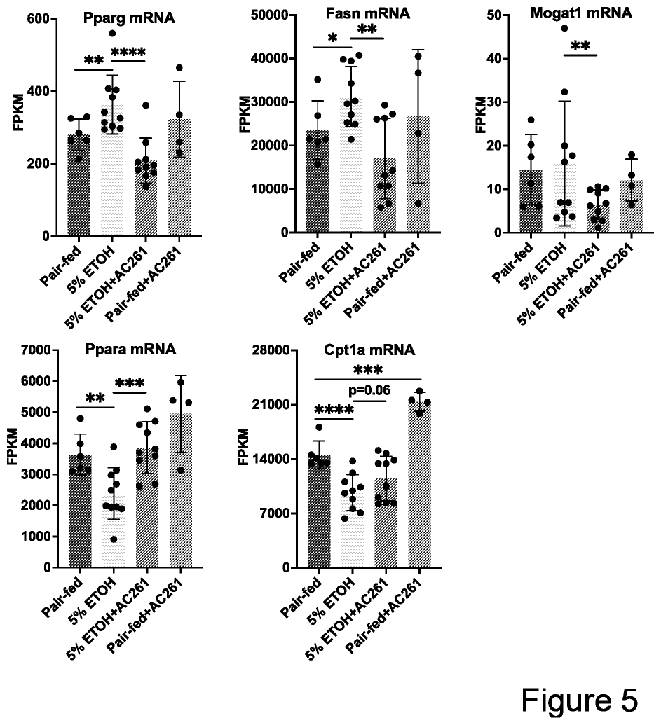

The mechanism by which AC261066 functions in lipid metabolism, including de novo lipogenesis and fatty acid β-oxidation in the liver, was assessed. RNA-seq data showed that compared to pair-fed mice, the livers from 5% ETOH-fed mice exhibited increases in mRNA levels of genes involved in de novo lipogenesis, including peroxisome proliferator-activated receptor gamma (Pparg), fatty acid synthase (Fasn), and monoacylglycerol o-acyltransferase 1 (Mogat1), and these increases were eliminated or attenuated by AC261066 treatment (FIG. 5).

Genes involved in fatty acid β-oxidation were also measured. Compared to Pair-fed mice, transcripts of genes involved in lipid β-oxidation in livers of 5% ETOH-fed mice, including peroxisome proliferator-activated receptor-α (PPARα; Ppara) and carnitine palmitoyltransferase-α (CPT1-α), were decreased, however, compared to ETOH-fed mice without AC261066, transcripts of these genes were increased in livers of 5% ETOH-fed plus AC261066 treated mice (FIG. 5).

Importantly, AC261066 did not significantly alter the hepatic mRNA levels of these genes, except CPT1-α, in Pair-fed mice (FIG. 5). The AC261066 induced increase in the mRNA level of CPT1-α in the Pair-fed condition suggests that AC261066 promotes fatty acid oxidation in the liver in its normal condition. These results indicate that the lipid lowering properties of RARβ₂ agonist AC261066 in ETOH fed mice involve transcriptional regulation of genes involved in both lipogenesis and fatty acid oxidation.

### Discussion

AC261066 modulates CYP2E1 and reduces progression of ALD. Retinoid and micronutrient deficiencies with chronic alcohol abuse contribute to the progression of liver damage and ALD. However, dietary and supplemental retinoids and other micronutrients are susceptible to depletions and catabolism with unchecked alcohol abuse. The data here show that 3 weeks of concomitant treatments with AC261 protected mice from steatosis and liver injury (FIGS. 2A-2C and FIGS. 3A-3B). Mechanistically, our data strongly suggest that the reduction in CYP2E1 expression in EtOH-AC compared to EtOH mice (FIGS. 4A, 4C) may be a key aspect of the anti-ALD properties of AC261, given that CYP2E1-overexpressing transgenic mice show higher levels of liver damage in ALD, and conversely, CYP2E1 mutant mice are protected from EtOH-driven steatosis, oxidative stress, and liver injury. Given that EtOH did not adversely impact hepatic AC261 levels and there was no evidence of hepatoxicity or fibrosis with concomitant administration of AC261 with EtOH, these data indicate the suitability of synthetic RARβ₂ agonists to keep ALD from progressing.

Conclusions. Given the unmet demand for anti-ALD drugs and growing evidence of the therapeutic potential of newer generation RAR-specific agonists, this Example supports a novel approach for prevention and treatment of ALD with synthetic agonists for RARβ₂.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method of treating, preventing, or ameliorating alcoholic liver disease (ALD) in a subject, comprising administering to said subject a therapeutically effective amount of a selective retinoic acid receptor-β₂ (RARβ₂) agonist or a pharmaceutically acceptable salt, ester, amide, prodrug thereof, or a combination thereof.

2. The method of claim 1, wherein the selective RARβ₂ agonist is AC261066 or AC55649.

3. The method of claim 1, wherein the selective RARβ₂ agonist is administered chronically.

4. The method of claim 1, wherein the selective RARβ₂ agonist is administered acutely.

5. The method of claim 1, wherein the selective RARβ₂ agonist is administered orally.

6. The method of claim 1, wherein the selective RARβ₂ agonist is administered parenterally.

7. The method of claim 1, wherein said liver disease is associated with reduced liver vitamin A levels.

8. The method of claim 1, wherein the subject is a human subject.

9. A method for inhibiting liver steatosis associated with alcohol consumption in a subject, comprising administering to said subject a therapeutically effective amount of a selective retinoic acid receptor-β₂ (RARβ₂) agonist or a pharmaceutically acceptable salt, ester, amide, prodrug thereof, or a combination thereof.

10. The method of claim 9, wherein the selective RARβ₂ agonist is AC261066 or AC55649.

11. The method of claim 9, wherein the selective RARβ₂ agonist is administered chronically.

12. The method of claim 9, wherein the selective RARβ₂ agonist is administered acutely.

13. The method of claim 9, wherein the selective RARβ₂ agonist is administered orally.

14. The method of claim 9, wherein the selective RARβ₂ agonist is administered parenterally.

15. The method of claim 9, wherein said liver steatosis is associated with reduced liver vitamin A levels.

16. The method of claim 9, wherein the subject is a human subject.

17. A method for inhibiting liver oxidative stress and/or hepatic lipogenesis associated with alcohol consumption in a subject, comprising administering to said subject a therapeutically effective amount of a selective retinoic acid receptor-β2 (RARβ₂) agonist or a pharmaceutically acceptable salt, ester, amide, prodrug thereof, or a combination thereof.

18. The method of claim 17, wherein the selective RARβ₂ agonist is AC261066 or AC55649.

19. The method of claim 17, wherein the selective RARβ₂ agonist is administered chronically.

20. The method of claim 17, wherein the selective RARβ₂ agonist is administered acutely.

21. The method of claim 17, wherein the selective RARβ₂ agonist is administered orally.

22. The method of claim 17, wherein the selective RARβ₂ agonist is administered parenterally.

23. The method of claim 17, wherein said liver oxidative stress and/or hepatic lipogenesis is associated with reduced liver vitamin A levels.

24. The method of claim 17, wherein the subject is a human subject.
